(19) United States
(12) Patent Application Publication
(10) Pub. No.: US 2007/0276193 A1
(43) Pub. Date: Nov. 29, 2007

(54) Vivo Diagnostic and Therapy Micro-Device

(76) Inventors: Florence Rivera, Tercis les bains (FR); Patrice Caillat, Grenoble (FR); Martine Cochet, Moirans (FR); Jean Berthier, Meylan (FR)

Correspondence Address: THELEN REID BROWN RAYSMAN & STEINER LLP, P. O. BOX 640640, SAN JOSE, CA, 95164-0640, US

(21) Appl. No.: 10/580,453

(22) PCT Filed: Nov. 19, 2004
(86) PCT No.: PCT/FR04/50602

§ 371 Date: Apr. 2, 2007

(30) Foreign Application Priority Data
Nov. 27, 2003 (FR) 03 50919
Mar. 4, 2004 (FR) 04 50446

(51) Int. Cl. A61B 5/00

(52) U.S. Cl. 600/300

(57) ABSTRACT

The invention relates to an in vivo diagnostic or therapy micro-device comprising a substantially longitudinal body provided with preferably parallel faces comprising, in the direction of its length, at least one main canal (24), one input (18) of which is located at a first end (14) of the body.

### TECHNICAL FIELD AND PRIOR ART

The invention relates to the domain of diagnostic and/or therapy micro-devices, for which applications are found in a wide variety of medical fields such as electrotransfection, electrostimulation, electrodiffusion, recording of the electrical or biochemical activity, or in vivo and in situ dispensing and sampling of substances.

Such micro-devices according to the invention are minimally invasive and can be used to investigate the human or animal body. They are diagnostic assistance tools or therapy assistance tools. They can be used to target areas with dimensions of between a few hundred micrometers and a few centimetres.

Imaging systems associated with different markers are known for functional in vivo monitoring of tissues of interest. Although the performance of these technologies is improving, they remain a global tool for study and diagnostic.

Some research laboratories have designed electrically addressable micro-injector prototypes. These devices have a thin end that can be inserted into the target tissue, and a thick end that can be used for electrical and fluid connections.

This second end is usually a few millimetres or a few centimetres wide and thick. It can be cumbersome and cannot be inserted in vivo which limits access to deep and fragile zones such as the brain. Therefore, these known devices are limited due to the size of the gripping element and connections.

Therefore the problem arises of making micro-devices for in vivo applications, particularly for a diagnostic and/or therapy.

The problem also arises of obtaining different functions in a device with a section or size of a few hundred micrometers.

### PRESENTATION OF THE INVENTION

The invention proposes to use other techniques for making implantable micro-devices. In particular, the invention proposes the use of microtechnological processes for catheter or probe type devices. Surprisingly, these micro-devices have proved their biocompatibility in vivo, even though the forms thus manufactured are not circular or even round.

The invention relates firstly to an in vivo diagnostic or therapy micro-device comprising:
a substantially longitudinal body provided with at least one main canal in the direction of its length, one input of which is located at a first end of the body,
and one or more secondary canals connected to at least one main canal and opening up sideways by lateral outputs.

Such a micro-device, for which the section may be provided with sharp or rounded corners and in particular may be quadrilateral shaped, can be used for easy injection of liquid products and/or microparticles in the human body, and particularly in the brain.

Such a device may also comprise one or more electrodes arranged on an outside portion of the body, and one or more electrical connection pins located at the first end of the body close to the input to said canal.

The invention also relates to an in vivo diagnostic or therapy micro-device comprising:
a substantially longitudinal body through which a main canal passes, for which one input is located at a first end of the body,
one or more electrodes located on an outside portion of the body,
one or more electrical connection pins located at the first end of the body, close to the input to said canal.

Once again, the section of the body of the micro-device may include sharp or rounded corners, for example it may be quadrilateral shaped.

In both embodiments described above, the electrical connection pins may comprise micro-cavities or etched areas made in the body of the micro-device.

These micro-cavities or etched areas may for example have a height and width between 10 μm and 50 μm.

Therefore the technological stack of the micro-device according to the invention, for example made of silicon, can be used to integrate the electrical and fluid connections stage.

Therefore, the dimensions of this stage are equivalent to the device itself and may be encased in a hollow guide device.

Preferably, a device according to the invention comprises a second bevel-shaped end.

It may also comprise two main parallel canals for the injection of different products or liquid products into the tissues.

One or more secondary canals may be connected to at least one main canal and may open up laterally through lateral outputs, which once again facilitates injection of product, or sampling of products, in the tissues passed through.

The body of the device may have a section with a maximum dimension of less than 1 mm, or a square or rectangular section in which each side has a maximum dimension of less than 300 μm or less than 900 μm.

For example, the longitudinal extension of the body itself is between 0.5 cm and 3 cm.

A funnel-shaped inlet into the fluid canal enables easy insertion of injection capillaries into the canal.

The invention also relates to a process for manufacturing an in-vivo diagnostic or therapy micro-device comprising:
the manufacture of two substantially longitudinal portions of the device, each portion comprising at least half a canal extending along a longitudinal direction, or a first portion comprising a canal,
the assembly of the two portions, directly to each other or with an intermediate layer, so as to form at least one main canal extending along a longitudinal direction.

A device according to the invention can thus be produced by using standard silicon techniques or silicon on insulator (SOI) type working techniques, these SOI techniques possibly being used for the manufacture of small micro-devices.

One or more electrodes, and one or more electrical connection pins, can be made on at least one of the two portions, for example by etching or by deposition of biocompatible metal.

The intermediate layer may comprise a fluid canal.

A portion of at least one secondary canal, or at least one complete secondary canal, may be made.

The invention also relates to a process for making an in vivo diagnostic or therapy micro-device comprising the manufacture of two half-devices in one or two SOI wafers, each wafer comprising a surface silicon layer with a free face, or first face, and a second face in contact with a buried insulating layer, this process comprising the following for each half-device:
etching of the first face of the silicon surface layer and deposit of a biocompatible noble metal on this first face, to make at least one electrode and at least one connection pin on it,
etching of the second face of the silicon surface layer to make at least one fluid half-network, comprising at least one half-canal extending along a longitudinal direction, and then
assembly of the two micro-devices through their second faces, possibly with an intermediate silicon layer, to form at least one fluid network canal.

### BRIEF DESCRIPTION OF THE FIGURES

FIGS. 1 to 4 represent various embodiments of the invention,

FIGS. 5A to 6 represent detailed embodiments of the proximal end of a device according to the invention,

FIGS. 7A to 11 represent steps in processes according to the invention.

### DETAILED PRESENTATION OF EMBODIMENTS OF THE INVENTION

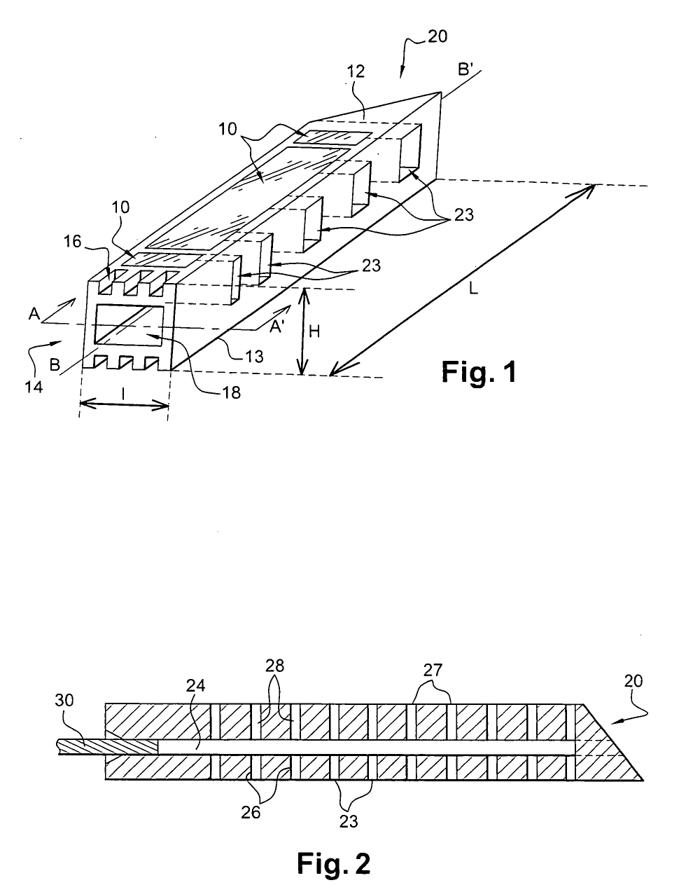

A first embodiment of the invention is illustrated in FIG. 1.

The micro-system in this Figure is substantially parallelepiped in shape. It has a substantially longitudinal extension, along a longitudinal axis BB′. Although the shape shown is parallelepiped, it is understood that it could be any elongated quadrilateral type of section, or even an arbitrary section with sharp corners, in other words non-rounded corners, or rounded corners. Preferably, and considering the manufacturing processes, the section of the micro-device is rectangular and/or the micro-device is plane, with two parallel longitudinal faces.

In the embodiment illustrated in FIG. 1, the micro-system has different electrodes 10 on its upper face 12 and on its lower face 13. It could also have electrodes on only one face.

These electrodes 10 can be individually addressed and electrically connected using connections 16 located on the proximal face 14 of the device. This face 14 also has an opening 18 to a fluid network.

As can be seen in FIG. 2 that shows a sectional view along plane AA′ in FIG. 1, such a fluid network is composed of a main canal 24 that serves secondary canals 26, 28.

The entry 18 to the main canal is located on the proximal face 14. One or more outputs 23, 27 of the secondary canals can be located on the lateral and/or upper 12 and/or lower 13 faces.

In the mode illustrated, the canal 24 does not open up on the side of the distal end 20 of the device. According to one variant, it could open up on the side of this end 20, as shown in continuous lines in FIG. 2.

According to another variant, the device may comprise only one main canal opening up on side 20 and no lateral canal, one or more electrodes being located on at least one of the outside faces of the device.

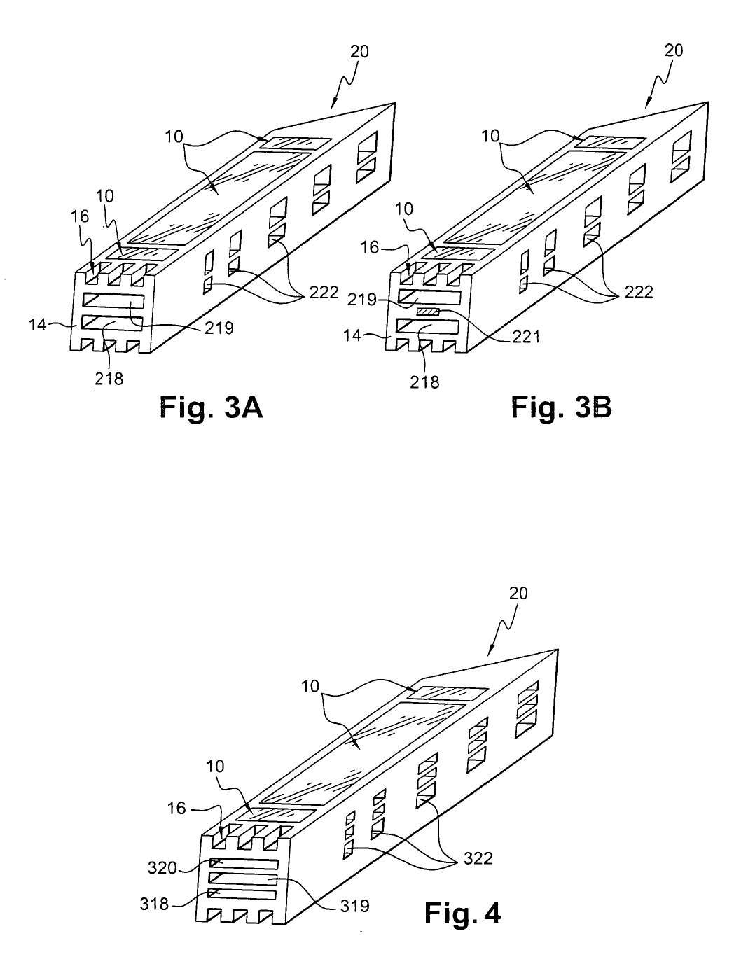

Several parallel fluid canals or networks can be made as illustrated in FIGS. 3A, 3B and 4; these figures represent a micro-device with two micro-fluidic networks (FIGS. 3A and 3B) and three micro-fluidic networks (FIG. 4).

Thus, FIGS. 3A and 3B show two inputs 218, 219 to fluid networks, and FIG. 4 shows three inputs 318, 319, 320 to such networks, these inputs being arranged in the proximal face 14 of the device. Such a device may or may not comprise lateral electrodes 10. One or more fluid canals may open up on the side of the distal end 20.

The section of the openings 18, 218, 219, 318, 319, 320 of the proximal face 14 varies as a function of the desired number of fluid networks and the required final size of the device. The number, sections and spacings between the fluidic outputs 22, 222, 322 of the secondary canals depend on the application. The angle formed between the secondary canals and the main canal may be between 0 and 90 degrees, for example between 10 and 90 degrees.

According to one variant, a device according to the invention comprises at least one main canal (two main canals in FIG. 3B) arranged as described above, opening up or not opening up on the side of the distal end, and a longitudinal wave guide 221 extending parallel to the axis of the device and the main canals, opening up on the side of the distal end 20, all with or without lateral electrodes 10.

The distal face 20 of the device is preferably bevelled to facilitate penetration of the device into a sensitive organ or tissue.

The height H and the width l of the proximal face are of the order of a few hundred micrometers each; for example, they may be between 100 μm and 300 μm, or 400 μm or 500 μm.

According to one example embodiment:
H=l=210 μm.

The length L of the device may for example be between 500 μm or 1 cm and 2 cm or 3 cm.

Slightly larger devices may be made for applications in parts of the body other than the brain, for example using standard silicon technologies and therefore less expensive, where H and l are each between 500 μm and 1000 μm or 1500 μm. Thus, for example:
H=900 μm and l=500 μm.

The micro-device is fixed at its proximal end 14 to a conventional insertion system so that it can be used. For example, it may be glued to a catheter or a probe; in particular it could be adapted to the end of a syringe.

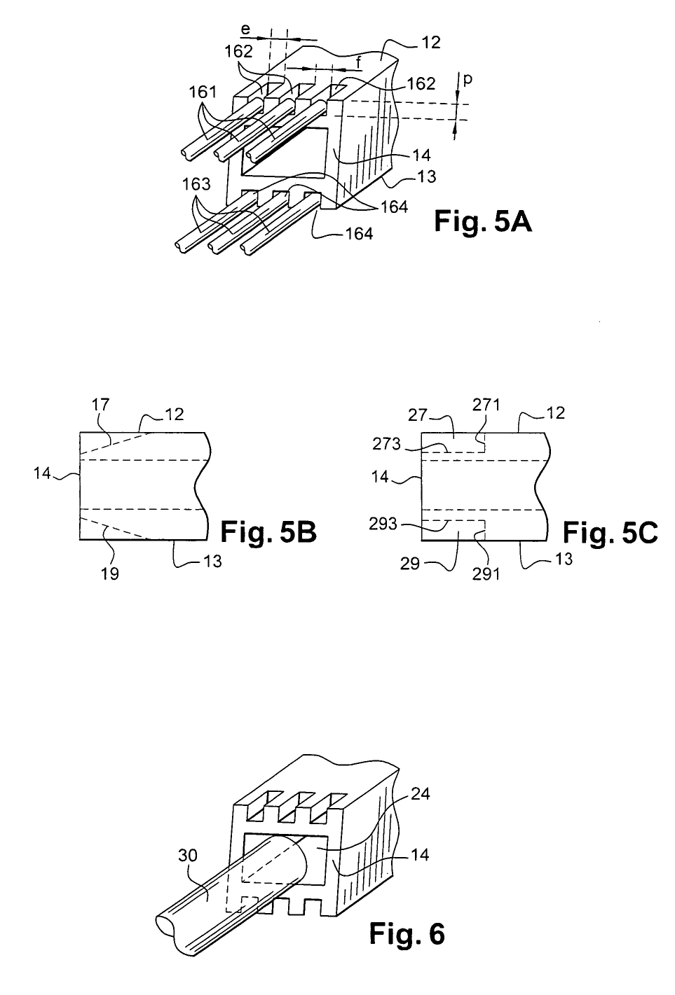

FIG. 5A more precisely shows the electrical connections stage 16. There are electrical connections 161, 163 on each side of the opening 18, for example cables inserted in notches 162, 164 specially provided for this purpose.

These notches are actually etched in at least one of the two faces 12-14; the two faces 12, 14 are etched in FIG. 5A, and both faces 13 and 14 are also etched.

The shape of the notches may be as shown in FIG. 5B; plane portions 17, 19 inclined from the upper faces 12 and the lower face 13 towards the proximal face 14, form contact areas.

Other forms are possible, for example parallelepiped shapes 27, 29 as illustrated in FIG. 5C.

A layer of biocompatible conducting metal may be placed on the plane portions 17, 19 or on the faces 271, 273 and 291, 293 of the parallelepiped shapes 27, 29 as described later, onto which the ends of connections 161, 163 will be fixed.

The dimensions e, f and p in FIG. 5A are the opening dimensions of electrical connection pins on the wafer surface. For example, each is between 30 μm and 50 μm or between 10 μm and 30 μm.

For extra cerebral applications for which dimensional constraints are less severe, as already indicated above, the values e, f and p may for example be between 30 μm and 100 μm, for example:
e=50 μm=f=p.

Therefore the micro-device according to the invention may have an integrated connection stage; electrodes 10 and the connections are located on the body of the device and in its prolongation, or in its periphery or its lateral walls, respectively, without projecting beyond or outside the cross-section (perpendicular to the longitudinal axis BB′) of the body. This enables insertion into guide systems of the type of those used in vivo and makes the device only very slightly destructive of tissues that it might encounter on its passage.

As illustrated in FIG. 6, a micro-capillary 30 for injection of a fluid may be inserted in the inlet to the main canal 24 of a micro-fluidic network. As can be seen in the top view in FIG. 2, the main canal inlet is then preferably a “V” canal so as to accommodate and guide a capillary 30 inserted through the proximal face 14 (see FIG. 6).

In the case of structures in FIGS. 3A, 3B and 4, each opening 218, 219, 318, 319, 320 can accommodate a capillary like that described above.

One of the main canals opening up on the side of the end 20 can hold an optical fibre, while another main canal will be used to circulate a fluid, for example injected through a capillary 30. Such a device may or may not comprise electrodes 10. The optical fibre can be used to inject or to collect radiation.

Therefore the technological stack of the micro-device according to the invention can be used to integrate the electrical and fluid connections stage.

Therefore, the dimensions of this stage are equivalent to the device itself and can be included in a hollow guide device.

A micro-device according to the invention can be used as an injector or an electrostimulator or an electrotransfector or an electrodiffuser.

Surface electrodes 10 can also be used to record the cellular electrical activity in response to a biochemical stimulation through the micro-fluidic injection network(s), or to record the cellular electrical activity at the same time as a liquid sample is taken through this (these) same fluidic network(s).

The electrodes of this device may also be biochemically functionalised so as to capture some cellular products of interest following injection or non-injection of bio-active molecules, an electrical measurement then being made. As an example, biochemical sensors or DNA or RNA segments or anti-bodies or cells can be fixed to these electrodes.

In a simpler embodiment, the device according to the invention does not include any means to make electrical measurements and therefore no electrodes 11 or electrical connecting pins 16, but it does have at least one longitudinal main canal and possibly one or more secondary canals and/or wave guides as described above. Such a fluidic system enables injection or sampling of product micro-quantities in the human body, and/or possibly sampling or injection of radiation.

Due to its size, and regardless of the planned embodiment, a device according to the invention can be used in cerebral structures without causing damage to the tissues encountered.

We will now describe a first manufacturing method. It makes use of “SOI” type techniques. For example, such techniques are described in the book by Q-Y Tong and U. Gösele entitled “Semi-conductor Wafer Bonding”, The Electrochemical Society & Series, 1999.

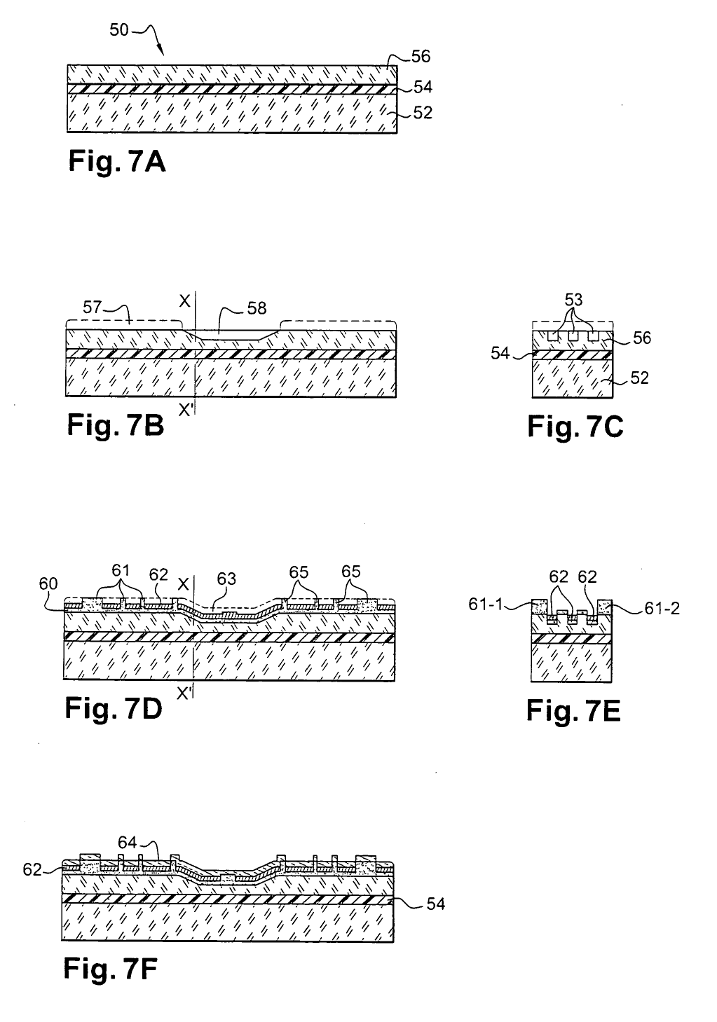

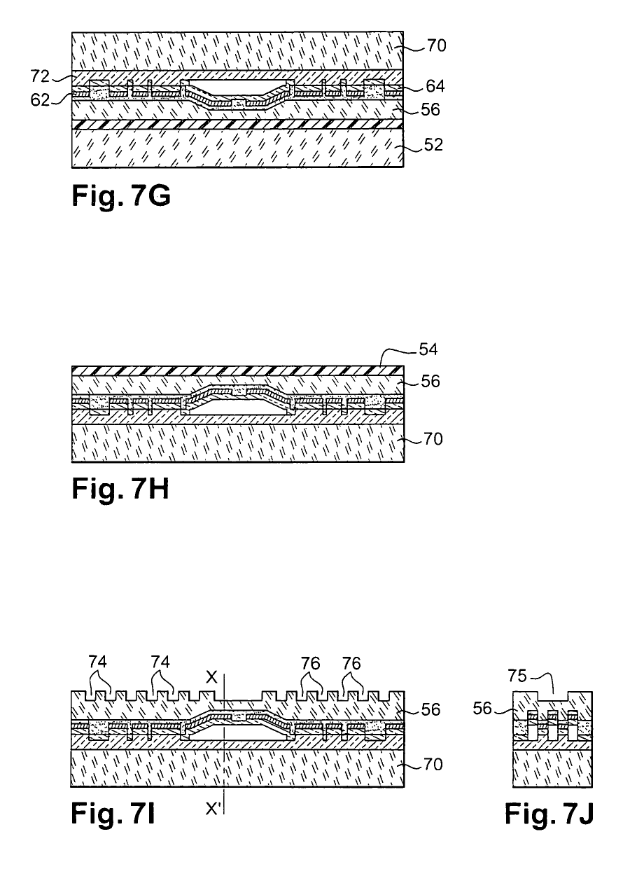

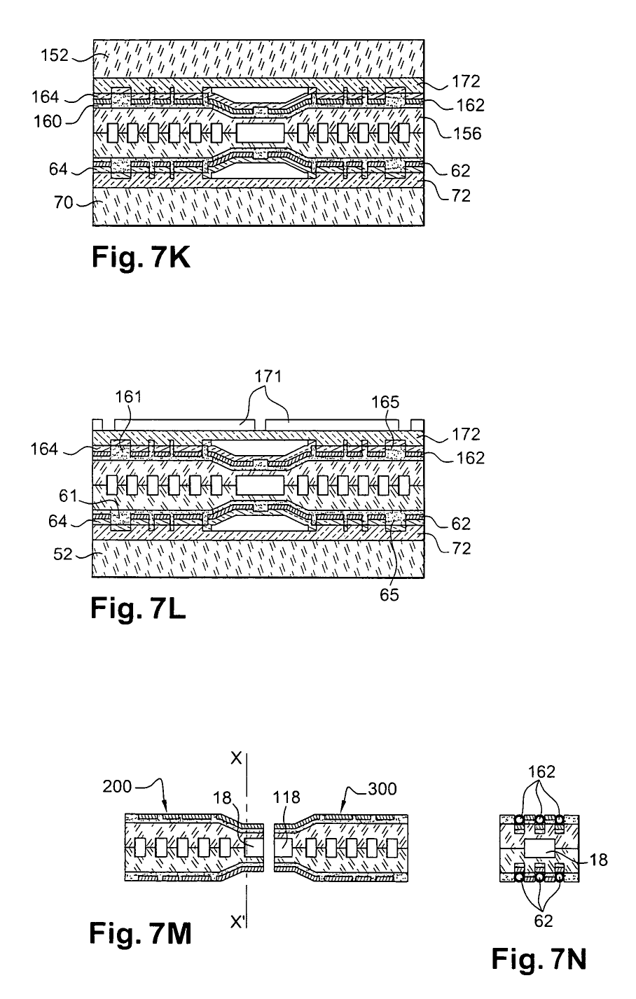

For example, an initial component 50 is an SOI substrate (FIG. 7A). An SOI (Silicon on Insulator) structure typically comprises a silicon layer 56 on which a buried layer 54 of silicon oxide is made, that itself is on top of a silicon substrate 52 that acts as a mechanical support. For example, such structures are described in FR-2 681 472.

Typically, the thickness of the layer 56 is between a few tens of micrometers, for example between 50 μm and 100 μm or 150 μm.

The thickness of the insulating layer 54 may be between 1 μm and a few tens of micrometers, for example 20 μm.

In a first step (FIG. 7B), notches 58 are made that prefigure electrical connection pins like those shown for example in FIGS. 5B and 5C. For example, these notches may be made by wet etching of silicon through an etched layer 57 of silicon nitride. This layer of silicon nitride is obtained by photolithography and then dry etching of a silicon nitride layer. The mask 57 is then removed.

FIG. 7C shows the appearance of the component obtained after this step, in a section along plane XX′ in FIG. 7B. The notches 53 obtained are shown in this Figure.

A layer 60 of silicon nitride (FIG. 7D) is then deposited followed by a layer 62 of a biocompatible noble metal (for example Au (gold) or Cr (chromium) or Ti (titanium) or Pt (platinum)). This metal layer is etched and the assembly is covered with a new layer 63 made of silicon nitride in which photolithography is applied to expose pins 61, 65 that will be used to isolate and delimit the different electrodes between themselves. The layer 63 is then eliminated, leaving the pins 61 and 65 behind.

FIG. 7E still shows plane XX′ displaying the structure obtained with a deposit of a metal layer 62 in the grooves 53, and on the non-etched plane area of the layer 56, and two lateral pins 61-1, 61-2 made of silicon nitride.

The assembly is then covered with an insulating layer 64, for example silicon oxide (FIG. 7F) and is then assembled with the surface layer 72 of silicon oxide of a component comprising a silicon substrate 70 (FIG. 7G) covered with the said layer 72 of silicon oxide. The assembly is made by molecular bonding at a temperature of about 300° C. The substrate 70 will then act as a support for subsequent operations.

The silicon substrate 52 is eliminated by polishing, leaving the insulating layer 54 behind (FIG. 7H).

The layers 54 and 56 are then etched to expose the canals 74, 76 of the future fluidic network (FIG. 7I).

FIG. 7J shows a section along axis XX′ showing a half 75 of the future longitudinal canal obtained by etching the layer 56.

The next step (FIG. 7K) is sealing of two symmetrical wafers by molecular bonding, the second wafer presenting a silicon layer 156 in which another fluidic half network has been etched, followed by a silicon nitride layer 160, a layer 162 of a biocompatible noble metal and two layers 164, 172 of an insulator (silicon oxide) on which a silicon substrate 152 is formed.

The substrate 152 is polished, and, through a mask 171, photolithography and dry etching of the layer 172 of silicon oxide, of pins 161, 165, of the subjacent layer of silicon nitride, and of the two half-bodies of the silicon device, and finally wet etching of the layers 64, 72 of silicon oxide lead to the release of two devices 200, 300 as illustrated in FIGS. 7L and 7M. In these Figures, the references 18 and 118 respectively denote the planned inlet for the fluidic network. FIG. 7N shows a lateral view along the XX′ plane showing the input 18 provided with electrical connection pins, particularly bearing metallic deposits 62, 162.

The result is thus a device conforming with FIG. 1.

A device like that shown in FIG. 3 that comprises two fluidic networks, is made by steps identical to those used in FIGS. 7I, 7J.

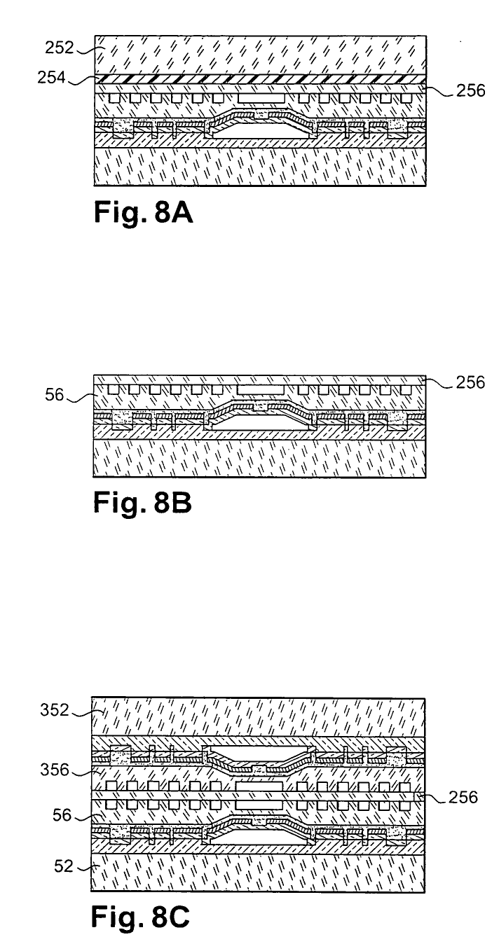

The component obtained is then assembled with an SOI wafer comprising a silicon layer 256, an insulating layer 254 and a silicon substrate 252 (FIG. 8A). This step is used to define a first fluidic network between the silicon wafers 56 and 256 (FIG. 8B). The substrate 252 and the insulating layer 254 are eliminated by polishing.

The component obtained is then assembled with a second component of the type illustrated in FIG. 7I with an etched silicon layer 356 to form a second fluidic network on it, with various layers of silicon nitride, biocompatible metal, silicon oxide on a substrate 352 (FIG. 8C) as already described above. The result is a structure formed with two fluidic networks separated by the silicon layer 256.

The following steps to enable release (polishing of substrate 352, photolithography, dry etching of silicon oxide, silicon nitride, silicon and finally dry etching of the layers 64, 72 of silicon oxide) are identical to or similar to those described above with reference to FIGS. 7L-7M.

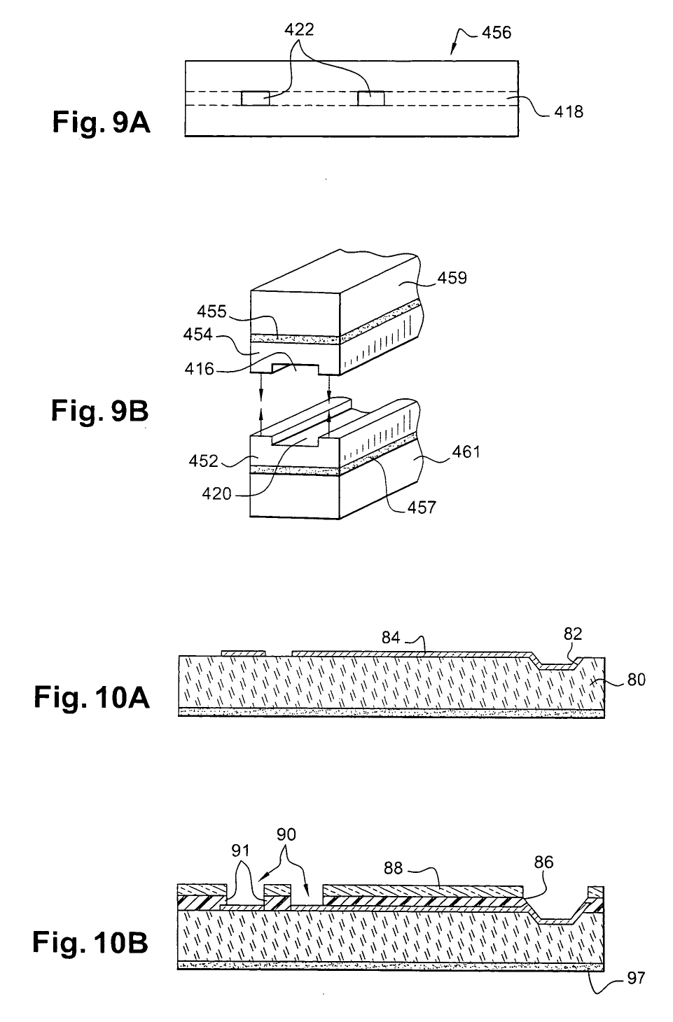

Manufacturing of a device like that in FIG. 4 comprising three fluidic networks uses a technique similar to the technique described above, except that wafer 256 is replaced by a component like that in FIG. 9A comprising a silicon wafer 456 inside which a canal 418 is made, and possibly secondary or lateral canals for which the lateral outputs 422 can be seen in FIG. 9A.

For example, this wafer is obtained by molecular assembly of two half-layers 452, 454 (FIG. 9B) of silicon in which two half-canals 416, 420 and the corresponding secondary half-canals were formed, these two wafers then being assembled as illustrated in FIG. 9B. Each of these wafers 452, 454 may be the silicon surface layer of an SOI component also comprising a substrate 459, 461 and an insulating layer 455, 457. The two SOI components are treated to make two half-canals 416, 420 in this surface layer and are then assembled as shown in FIG. 9B. The substrate 459 and the insulating layer 455 are then eliminated, the substrate 461 being kept temporarily to enable transfer as illustrated in FIG. 8A.

Intermediate wafers 456 can be assembled or stacked, with one intermediate wafer for each main canal along the longitudinal axis BB′ of the device.

The subsequent steps of the process, until the components are released, are identical or similar to those described above.

Steps similar to those in FIGS. 9A and 9B can be used to form a longitudinal wave guide, rather than a canal 418 and secondary canals. For example silica is deposited or formed in the two half-canals 416, 420, the two components 454, 452 then being assembled as described above. The result can thus be a structure like that shown in FIG. 3B.

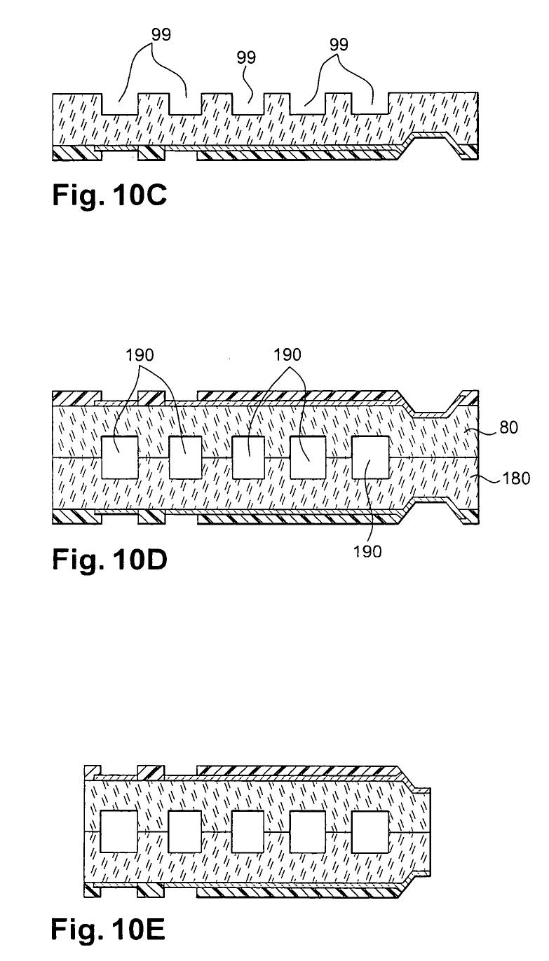

FIGS. 10A-10E illustrate a process for manufacturing a slightly larger device with standard silicon technologies. This process is particularly suitable for making a device like that already mentioned above, for which the width l and the height H are for example between 500 μm and 900 μm.

A cavity 82, which will form the electrical connection pins, is made on a silicon wafer 80 for example with a thickness of between 250 μm and 500 μm, this cavity is obtained by wet etching of silicon 80 through a silicon nitride mask with an appropriate shape.

A deposit of a layer 84 of a noble and/or biocompatible metal is then made after passivation by the deposition of a silicon oxide layer. This layer 84 is etched either by wet or dry etching through a resin mask (not shown in FIG. 10A).

A silicon oxide layer 86 is then deposited. This layer is etched through a resin mask, this step being used to expose openings 90 and to define pins 91 between the different electrodes. In FIG. 10B, the reference 88 denotes a mask, for example made of resin or metal.

The next step (FIG. 10C) is etching on the back face of the silicon wafer 80, so as to make half canals and lateral openings 99 that will define the fluidic network. This etching is obtained by dry etching through a mask, for example a resin mask, formed on a layer 97 of a silicon nitride deposited on the back face (FIG. 10B).

Two components thus obtained are then assembled as illustrated in FIG. 10D. In this Figure, the reference 180 denotes the second silicon wafer in which the second half-component is made. The lateral openings 190 of the fluidic network can also be seen.

A cutting step, implemented using dry etching techniques already described above, is then used to release the device (FIG. 10E).

Once again, the number of canals can be increased using techniques similar to those described above with reference to FIGS. 8A-8C and 9A-9B.

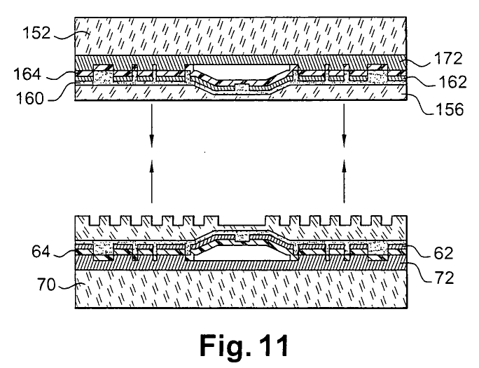

According to one variant of the process shown in FIGS. 7A-7N, a complete fluidic network is made rather than two half-devices each having a half-fluidic network which are then assembled. For example (FIG. 11), the layer 56 in FIG. 7I is etched more deeper so that the component obtained has to be assembled with a component in which the layer 156 has not been etched, and not with an identical component as shown in FIG. 7K. Subsequent steps leading to the release of components 200, 300 are similar to what has already been described. This variant may also be combined with the variants in FIGS. 8A-8C and 9A-9B. It may also apply to the process in FIG. 10A-10E: in this process, the device may be made by assembly of a component similar to that in FIG. 10C, etched to form a fluidic network with a second component that is not etched to form such a network.

In all the processes described above, deposits of silicon nitride are made by LPCVD (Low Pressure Chemical Vapour Deposition) and deposits of silicon dioxide are made by PECVD (Pressure Enhanced Chemical Vapour Deposition) or by thermal oxidation.

Manufacturing techniques that can be used within the scope of the invention are also described in the book by S Wolf et al. “Silicon Processing, Vol. 1: Process technology”, Lattice press, California, 1986, and particularly p. 161-197, 407-513, 532, 539-585 and in the book “VSLSI Technology”, Ed. SM Sze, McGraw Hill International Editions, Electrical & Electronic Engineering Series”, 1988, particularly p. 375-421.

A micro-system according to the invention can be used either to obtain information about small target structures, or to diagnose some pathologies or functions through electrical, electrochemical or biochemical sensors, or to treat or inhibit some pathological zones by electrostimulation and/or the release of active substances in situ.

1. In vivo diagnostic or therapy micro-device comprising:
a substantially longitudinal body having a quadrilateral-shaped section, provided with at least one main canal in the direction of its length, one input of which is located at a first end of the body,
and several secondary canals connected to at least one main canal and opening up sideways by lateral outputs.

2. Micro-device according to claim 1, further comprising:
one or more electrodes arranged on an outside portion of the body,
one or more electrical connection pins located at the first end of the body close to the input to the said canal.

3. Micro-device according to claim 2, the electrical connection pins comprising micro-cavities made in the body of the micro-device, the cavities having preferably a height and width between 10 μm and 50 μm.

4. Micro-device according to claim 1, comprising at least two parallel main canals.

5. Micro-device according to claim 1, at least one of the main canals opening up to a second end of the body, called the distal end, and the inlet into at least one main canal being funnel-shaped.

6. Micro-device according to claim 1, the body having two parallel opposite surface areas between the first and the second ends, and comprising a second bevel-shaped end.

7. Micro-device according to claim 1, the body having a square or rectangular section in which each side has a maximum dimension of less than 900 μm, preferably less than 300 μm, and the longitudinal extension of the body being preferably between 0.5 cm and 3 cm.

8. Micro-device according to claim 1, the body of the device being made of silicon.

9. Micro-device according to claim 1, further comprising a wave guide.

10. In vivo diagnostic or therapy micro-device comprising:
a substantially longitudinal body with a quadrilateral-shaped section, provided with at least one main canal in the direction of its length, one input of which is located at a first end of the body,
one or more electrodes located on an outside portion of the body,
one or more electrical connection pins located at the first end of the body, close to the input to said canal.

11. Micro-device according to claim 10, the electrical connection pins comprising micro-cavities made in the body of the micro-device, the micro-cavities having preferably a height and width between 10 μm and 50 μm.

12. Micro-device according to claim 10, comprising at least two parallel main canals.

13. Micro-device according to claim 10, at least one of the main canals opening up to a second end of the body, called the distal end, and the inlet into at least one main canal being funnel-shaped.

14. Micro-device according to claim 10, the body having two parallel opposite surface areas between the first and the second ends, and comprising a second bevel-shaped end.

15. Micro-device according to claim 10, the body having a square or rectangular section in which each side has a maximum dimension of less than 900 μm, preferably less than 300 μm, and the longitudinal extension of the body being preferably between 0.5 cm and 3 cm.

16. Micro-device according to claim 10, the body of the device being made of silicon.

17. Micro-device according to claim 10, further comprising a wave guide.

18. Process for manufacturing an in-vivo diagnostic or therapy micro-device from silicium comprising:
the manufacture of two substantially longitudinal portions of the device, each portion comprising at least half a canal extending along a longitudinal direction of the micro-device, or a first portion comprising a canal extending along the longitudinal direction of the micro-device,
assembly of the two portions, directly to each other or with an intermediate layer, so as to form at least one so-called main canal extending along the longitudinal direction.

19. Process according to claim 18, further comprising the production of one or more electrodes and one or more electrical connection pins on at least one of the two portions.

20. Process according to claim 19, the electrode(s) and the connection pin(s) being obtained by etching or by deposition of biocompatible metal.

21. Process according to claim 18, each of the portions being made in a silicon surface layer of an SOI substrate.

22. Process according to one of claim 18, comprising an intermediate layer itself being provided with a fluidic canal.

23. Process according to claims 18, further comprising the manufacture of at least one secondary canal portion, connecting to the half-canal or the main canal, the assembly of the two portions of the body forming at least one secondary canal connecting to the main canal.

24. Process according to claim 18, further comprising a step for making an optical guide.
